(19) United States
(12) Patent Application Publication
(10) Pub. No.: US 2020/0338082 A1
(43) Pub. Date: Oct. 29, 2020

(54) FORMULATIONS, SYSTEMS, AND METHODS FOR THERAPEUTIC TREATMENT

(71) Applicant: Scott ACKLER, Gurnee, IL (US)

(72) Inventor: Scott ACKLER, Gurnee, IL (US)

(21) Appl. No.: 16/959,046

(22) PCT Filed: Dec. 28, 2018
(86) PCT No.: PCT/US2018/067827

§ 371 Date: Jun. 29, 2020

Related U.S. Application Data
(60) Provisional application No. 62/611,294, filed on Dec. 28, 2017.

(51) Int. Cl. A61K 31/519; A61K 31/337; A61K 47/10; A61K 47/14; A61K 31/7048; A61K 47/18; A61K 31/475; A61M 5/142

(52) U.S. Cl. CPC A61K 31/519; A61K 31/337; A61K 47/10; A61K 47/14; A61K 9/0019; A61K 47/18; A61K 31/475; A61M 5/142; A61K 31/7048

(57) ABSTRACT

Formulations for the delivery of therapeutic agents, including chemotherapeutic agents, are provided. In these formulations, a concentrated therapeutic agent and an excipient are configured to be delivered directly by a catheter to a subject in need of therapeutic treatment. Also provided are systems for therapeutic treatment comprising a concentrated therapeutic formulation and a delivery device, where the system is configured for direct delivery of the concentrated formulation by a catheter to a subject in need of therapeutic treatment. Methods of therapeutic treatment comprising delivering a concentrated therapeutic formulation to a subject in need of treatment are also provided, where a concentrated therapeutic agent is delivered directly to the subject by a catheter using a delivery device.

### CROSS-REFERENCE TO RELATED APPLICATIONS

This application claims the benefit of U.S. Provisional Application No. 62/611,294, filed on Dec. 28, 2017, the disclosure of which is incorporated herein by reference in its entirety.

### BACKGROUND OF THE INVENTION

Drug delivery devices capable of the precise and reliable delivery of desired doses of a beneficial agent over extended periods of time are known in the art. For example, such devices are well known in the field of diabetes, where external insulin pumps have been developed that can provide reliable delivery of insulin over long periods. Insulin pumps generally consist of a pump mechanism, an insulin container, a processor, and a power source for the processor and pump mechanism. The pump mechanisms generally use motor driven push rods to drive a piston into the insulin containment region of the insulin container, thus forcing the insulin into a delivery tube and thereby into the patient. The motor driven push rod and piston assemblies of known insulin pumps provide drug delivery that is accurate, reliable, and space efficient. Exemplary insulin pumps are described in U.S. Pat. Nos. 5,097,122; 5,505,709; 5,637,095; and 6,248,093.

Implantable delivery devices have also been developed. Such devices include, for example, osmotically driven pumps capable of delivering an active agent formulation, such as a solution or a suspension, at a desired rate over an extended period of time. Time periods for delivery may range from a day, a week, a month, or even a year or more. Other exemplary implantable devices include regulator-type implantable pumps that provide constant flow, adjustable flow, or programmable flow of beneficial agent formulations. Exemplary devices are available from Codman of Raynham, Mass., Medtronic of Minneapolis, Minn., and Tricumed Medinzintechnik GmbH of Germany Further examples of implantable devices are described in U.S. Pat. Nos. 5,511,355; 5,836,935; 5,976,109; and 6,283, 949.

Since these devices can be designed to deliver a desired active agent at therapeutic levels over an extended period of time, external and implantable delivery systems can advantageously provide long-term therapeutic dosing of a desired active agent without requiring frequent visits to a healthcare provider or repetitive self-medication. Therefore, external and implantable delivery devices can work to provide increased patient compliance, reduced irritation at the site of administration, fewer occupational hazards for healthcare providers, reduced waste hazards, increased therapeutic efficacy, and improved safety profile through enhanced dosing control.

In the case of delivery of chemotherapeutic agents, reliable delivery using such devices could enable effective treatment of patients without requiring that the patients spend extensive periods of time at infusion centers, thus increasing quality of life for the treated patient and decreasing costs for treatment. Further, continuous delivery eliminates the peak-trough phenomenon seen with episodic dosing, providing efficacy without subsequent toxicity associated with these agents. The approach thus has the dual advantage of lowering the cost of therapy, by minimizing or eliminating hospitalizations associated with chemotherapy-induced side effects, and improving response rates, by increasing the percentage of patients who can safely complete therapeutic regimens.

Chemotherapeutic agents, as well as many other pharmaceutically active therapeutic agents, frequently have low solubility in the solutions used for their delivery, thus requiring large volumes of infusion of the typical delivery solution in order to obtain therapeutically effective levels of the agents in a patient's bloodstream. Diluted chemotherapeutic agents must therefore be delivered over extended periods of time, in unpleasant, impersonal, and potentially unhygienic hospital-based or clinic-based infusion centers.

Delivery of chemotherapeutic agents by continuous infusion has been reported in a small number of studies with some success, but it is not clear that the approaches can be scaled for use in larger patient populations, with a broader range of therapeutic agents, over longer periods of time, and with reliable and effective outcomes. For example, paclitaxel was continuously delivered, either with or without radiation, over a 7 week period in a phase I clinical trial to patients with locally advanced non-small cell lung cancer. Rosenthal et al. (2006) J. Thorac. Oncol. 1:38-45. The study demonstrated the ability to deliver large cumulative doses of paclitaxel at levels known to be associated with radiosensitization of patients and established the safety of such long-term continuous treatments but used a diluted form of paclitaxel and thus still required the delivery of large volumes of drug.

Another phase I study demonstrated the continuous delivery over 96 hours of paclitaxel or doxorubicin in patients with relapsed epithelial ovarian cancer. Duska et al. (1999) Clin. Cancer Res. 5:1299. The study established plasma levels and defined principal toxicities for each drug and determined the maximum tolerated doses of the drugs but did not extend treatments for long time periods or provide the drugs in more highly concentrated formulations. In a study of childhood leukemia patients, the continuous infusion of doxorubicin did not provide a cardioprotective advantage over bolus infusion of the drug, but the infusion was carried out for only 48 hours, and the drug was not administered at higher than normal concentrations. Lipshultz et al. (2002) J. Clin. Oncol. 20:1677.

In yet another phase I study, topotecan was administered to patients with recurrent ovarian cancer by ambulatory infusion pump via venous access device for 21-day cycles. Hochster et al. (1999) J. Clin. Oncol. 17:2553. Patients were evaluated for toxicity, and after appropriate dose modifications, were treated with subsequent 21-day infusion cycles. Although the drug was administered at normal concentrations and thus required relatively large infusion volumes, the prolonged infusion regimen was found to be well tolerated by patients. A subsequent phase II clinical study examined the toxicity and efficacy of combined therapy with cisplatin and the prolonged infusion of topotecan in ovarian cancer patients. Hochster et al. (2006) Gynecol. Oncol. 100:324. Although the initial regimen was not well tolerated by patients, a lowered dose and duration of topotecan (0.3 mg/m²/day infused continuously over 14 days) was better tolerated.

Irinotecan has also been tested in a phase I study of metastatic colorectal cancer patients treated by a 7-day continuous infusion of the drug every 21 days. Masi et al. (2004) Clin. Cancer Res. 10:1657. This study demonstrated biological effects of the drug on patients treated by prolonged infusion, even though total doses were significantly lower than those administered by a standard 30-90 minute infusion. Longer periods of infusion or the use of a more highly concentrated drug were not reported.

A combination phase II regimen for the treatment of metastatic colorectal cancer including the continuous infusion of 5-fluorouracil by disposable pump in outpatients has also been reported. André et al. (1999) Eur. J. Cancer 35:1343. The method consisted of an initial 90-minute infusion of irinotecan in combination with a 2-hour infusion of leucovorin, a bolus treatment with 5-fluorouracil, and finally a 46-hour continuous infusion with 5-fluorouracil. Longer periods of infusion were not reported.

Finally, patients with advanced malignancies have been treated with a prolonged continuous intravenous infusion of vinblastine by implantable pump. Ratain and Vogelzang (1986) Cancer Res. 46:4827. In these phase I studies, vinblastine was dissolved in an aqueous sodium chloride solution for use in the pump. The studies demonstrated the feasibility of prolonged infusions of vinblastine and established steady-state concentrations of drug and dosing rates suitable for phase II trials.

The ability to provide therapeutic agents to patients without the need to visit an infusion center provides many advantages. Such an option would be significantly more affordable and efficient than current treatment options in infusion centers or other sites of care, would limit the exposure of patients to hospital-based or clinic-based infectious agents, and would be far more patient friendly than requiring a patient to spend long periods of time receiving therapeutic treatment in an infusion center.

There is accordingly a need to develop new formulations, systems, and methods for therapeutic treatment that will address the deficiencies of the existing technologies. Ideally, such formulations, systems, and methods will allow for the delivery of therapeutic agents from an external or implantable device at a variety of controlled rates over long periods of time and will maintain the stability of the therapeutic agents over extended periods of time and under a variety of conditions.

### SUMMARY OF THE INVENTION

The present disclosure addresses these and other needs by providing in some aspects therapeutic formulations comprising:

a therapeutic agent and an excipient,

wherein the formulation is suitable for direct delivery at high concentration by a catheter to a subject in need of therapeutic treatment.

In some embodiments, the therapeutic agent is a chemotherapeutic agent. Specifically, the chemotherapeutic agent can be an alkylating agent, an anthracycline, a cytoskeletal disruptor, an epothilone, a histone deacetylase inhibitor, a kinase inhibitor, a nucleotide analogue or precursor, a peptide, a platinum-based agent, a retinoid, a topoisomerase inhibitor, or a vinca alkaloid. More specifically, the chemotherapeutic agent can be paclitaxel, taxol, docetaxel, taxotere, cisplatin, carboplatin, oxaliplatin, etoposide, vincristine, cyclophosphamide, methotrexate, fluorouracil, gemcitabine, topotecan, irinotecan, melphalan, or doxorubicin.

In embodiments, the excipient increases the solubility of a hydrophobic therapeutic agent in the formulation. For example, the excipient can comprise dimethylacetamide, ethanol, a polyethylene glycol, propylene glycol, a polyethoxylated nonionic surfactant, a polysorbate nonionic surfactant, or a cyclodextrin.

In other embodiments, the excipient increases the solubility of a hydrophilic therapeutic agent in the formulation. For example, the excipient can comprise an acid, a base, or a salt.

In specific embodiments, the formulation is suitable for direct delivery to the subject without dilution, for example direct delivery to the subject without dilution into an aqueous solution.

The disclosure provides in other aspects systems comprising:

any of the above-described formulations; and

a delivery device,

wherein the system is configured for direct delivery of the formulation by a catheter to a subject in need of therapeutic treatment.

More specifically, the delivery device can be a precision pumping device.

In some embodiments, the formulation can be delivered from an exchangeable reservoir, can be configured to deliver the formulation to the subject at no more than 1 mL per hour, can be configured to deliver at least a therapeutic dose of the therapeutic agent to the subject for at least 12 hours, can be configured to deliver less than a toxic dose of the therapeutic agent to the subject for at least 12 hours, or can be configured to deliver at least a therapeutic dose and less than a toxic dose of the therapeutic agent to the subject for at least 12 hours.

In some embodiments, the catheter can be a peripherally inserted central catheter, a Hickman line, a Broviac catheter, a Groshong catheter, or a central venous catheter.

In embodiments, the delivery can be by steady-state delivery and/or can reduce the incidence of neutropenia in the subject.

In preferred embodiments, the subject can be a human subject.

In still other aspects, the disclosure provides methods of treatment comprising the steps of:

delivering a therapeutic formulation to a subject in need thereof, wherein

the therapeutic formulation is any of the above formulations, and the formulation is delivered directly to the subject by a catheter using a delivery device.

### BRIEF DESCRIPTION OF THE DRAWINGS

FIG. 1. Summary of the solubility of various chemotherapeutic agents in various excipients.

### DETAILED DESCRIPTION OF THE INVENTION

### Formulations for the Delivery of Therapeutic Agents

The instant disclosure provides in one aspect formulations useful in therapeutic treatments, particularly in the delivery of therapeutic agents to patients in need of such treatment over long periods of time in reliable and precise dosages. The formulations are especially suitable for direct delivery by a catheter to a subject in need of therapeutic treatment.

Because many of the most commonly used therapeutic agents, including in particular chemotherapeutic agents and other useful pharmaceutically active therapeutic agents, are poorly soluble molecules, it can be difficult to achieve sufficiently high concentrations of these agents in the vehicles used for their delivery and can therefore be difficult to achieve therapeutically effective doses of the agents when administered to subjects in need of treatment. The agents are therefore typically formulated with solubilization agents that increase the solubility of the drug in the delivery vehicle. The agents routinely utilized in solubilizing drugs for oral and injectable delivery include pH modifiers, water-soluble organic solvents, surfactants, water-insoluble organic solvents, medium- and long-chain triglycerides, cyclodextrins, and phospholipids. See, e.g., Strickley (2004) Pharma. Res. 21:201. Of importance in any successful solution formulation are solubility and stability. Of further importance in such formulations is the ability of the therapeutic agent to be rapidly and efficiently diluted and mixed with the blood stream of the subject upon delivery to the subject. The therapeutic formulations of the instant disclosure address these and other issues by providing soluble and stable formulations comprising a therapeutic agent and an excipient, where the excipient increases the solubility of the therapeutic agent in the formulation. The formulations are suitable for direct delivery of the therapeutic agent at high concentration by a catheter to a subject in need of therapeutic treatment, such that the agent is rapidly and efficiently diluted and mixed into the bloodstream of the subject upon delivery. The formulations thus enable the efficient and reliable delivery of therapeutically effective concentrations of the therapeutic agent to the subject over long periods of time with relatively small volumes of delivery vehicle. By providing a constant rate of delivery of the active therapeutic agent, the formulations also provide a more consistent delivery of the agent, thus minimizing any possible toxic effects of the agent, even where the therapeutic window for the therapeutic agent is narrow.

In some embodiments, the excipient increases the solubility of a hydrophobic therapeutic agent in the formulation, thus providing a highly concentrated form of the agent. For example, the excipient may comprise dimethylacetamide, ethanol, a polyethylene glycol, propylene glycol, a polyethoxylated nonionic surfactant, a polysorbate nonionic surfactant, or a cyclodextrin. Such excipients are well suited for increasing the solubility of hydrophobic therapeutic agents in the delivery vehicle and thus for increasing the solubility and accordingly the concentration of the therapeutic agent in the formulation.

In some embodiments, the excipient increases the solubility of a hydrophilic therapeutic agent in the formulation. For example, in some embodiments, the excipient comprises an acid, a base, a salt, or another suitable agent for increasing the solubility of the hydrophilic therapeutic agent in the formulation.

As noted above, the formulations of the instant disclosure are suitable for direct delivery at high concentration to a subject in need of therapeutic treatment. Such formulations typically comprise concentrations of a therapeutic agent that are higher than the concentrations normally used in the art to deliver the respective therapeutic agent to the subject. For example, the formulation may comprise a therapeutic agent dissolved in an excipient at a concentration of at least 1 mg/mL. More specifically, the formulation may comprise a therapeutic agent dissolved in the excipient at a concentration of at least 3 mg/mL, at least 5 mg/mL, at least 10 mg/mL, at least 30 mg/mL, at least 50 mg/mL, or even higher.

In some embodiments, the therapeutic agent is a chemotherapeutic agent. For example, the chemotherapeutic agents usefully provided in the instant formulations can include, without limitation, any agent that is effective in the treatment of any cancer in a mammalian subject. Such agents include, for example, alkylating agents, anthracyclines, cytoskeletal disruptors (e.g., taxanes), epothilones, histone deacetylase inhibitors, kinase inhibitors, nucleotide analogues and precursors, peptides, including antimicrobial peptides, platinum-based agents, retinoids, topoisomerase inhibitors, and vinca alkaloids and their derivatives. Many of these agents have relatively low solubility in aqueous solution, however, such that they normally need to be administered by infusion of large volumes of dilute aqueous solutions. Such chemotherapeutic agents will dissolve at much higher concentrations in a suitable excipient, however, as described in detail herein.

In preferred embodiments, the chemotherapeutic agent of the instant formulations is paclitaxel, taxol, docetaxel, taxotere, cisplatin, carboplatin, oxaliplatin, etoposide, vincristine, cyclophosphamide, methotrexate, fluorouracil, gemcitabine, topotecan, irinotecan, melphalan, or doxorubicin. More preferably, the chemotherapeutic agent is paclitaxel, docetaxel, etoposide, methotrexate, or vinblastine.

The formulations of the instant disclosure also comprise one or more excipients, which may also be described herein as a “vehicle” or a “solvent”. Although it is contemplated that any non-toxic excipient could serve as the excipient of the instant formulations, the excipients of the instant disclosure are preferably excipients that have already been approved by an appropriate reviewing authority (e.g., the U.S. Food and Drug Administration) for use in parenteral pharmaceutical formulations. It should also be understood that although the instant formulations typically do not contain any added water, the presence of water in the formulations is not necessarily precluded, whether by direct addition of water to the formulations or by the absorption of water vapor from the atmosphere due to hygroscopicity of components of the formulations.

In embodiments, the excipient comprises dimethylacetamide (DMA), ethanol, a polyethylene glycol (PEG), propylene glycol, a polyethoxylated nonionic surfactant, such as a polyethoxylated castor oil or a polyethoxylated stearic acid, a polysorbate nonionic surfactant, or a cyclodextrin.

More specifically, in some embodiments, the excipient of the instant formulations comprises a polyethylene glycol. Suitable polyethylene glycols are readily available from commercial sources. Exemplary polyethylene glycols include polyethylene glycol 300 (PEG300), polyethylene glycol 400 (PEG400), and polyethylene glycol 4000 (PEG4000). In preferred embodiments, the polyethylene glycol is PEG300 or PEG400.

In other embodiments, the excipient of the instant formulations comprises a polyethoxylated nonionic surfactant. Exemplary polyethoxylated nonionic surfactants include the polyethoxylated castor oils, such as, for example, the commercially available Cremophor® EL, Cremophor® ELP, and Cremophor® RH40 (also known as Kolliphor® EL, Kolliphor® ELP, and Kolliphor® RH40, respectively), which are available from the BASF Corp. or Sigma-Aldrich. Other exemplary polyethoxylated nonionic surfactants include the polyethoxylated stearic acids, such as, for example, the commercially available Solutol® HS-15 (also known as Kolliphor® HS 15), which is also available from the BASF Corp. or Sigma-Aldrich.

In still other embodiments, the excipient of the instant formulations comprises a polysorbate nonionic surfactant. Exemplary polysorbate nonionic surfactants include the Tween® surfactants, such as Tween® 20 and Tween® 80 (also known as polysorbate 20 and polysorbate 80, respectively). These excipients are also widely available from commercial sources.

In yet still other embodiments, the excipient of the instant formulations comprises a cyclodextrin, such as an α-, β-, or γ-cyclodextrin.

In preferred embodiments, the excipient of the instant formulations is an excipient that has been used in formulations previously approved for pharmaceutical use by the U.S. Food and Drug Administration. Accordingly, in these embodiments, the excipient of the instant formulations is preferably dimethylacetamide, ethanol, polyethylene glycol 400, propylene glycol, a Cremophor® (or a Kolliphor®), a Tween®, a Solutol®, or a cyclodextrin.

The excipients of the disclosure may be included in the instant formulations either alone or in any combination. Preferably the formulations comprise one, two, three, or even more different excipients. In some preferred embodiments, the formulations comprise a polyethoxylated castor oil and a polyethylene glycol, for example Cremophor® EL or Cremophor® ELP and PEG400. In other preferred embodiments, the formulations comprise a polyethoxylated stearic acid and a polyethylene glycol, for example Solutol® HS-15 and PEG400. In yet other preferred embodiments, the formulations comprise a polyethylene glycol and a polysorbate nonionic surfactant, for example PEG400 and Tween® 20 or Tween® 80. In still other preferred embodiments, the formulations comprise a polyethoxylated castor oil, a polyethylene glycol, and dimethylacetamide, for example Cremophor® EL or Cremophor® ELP, PEG400, and dimethylacetamide. In still yet other preferred embodiments, the formulations comprise a polyethoxylated stearic acid, a polyethylene glycol, and dimethylacetamide, for example Solutol® HS-15, PEG400, and dimethylacetamide.

The excipients of the instant formulations can be combined in various ratios, as would be understood by those of ordinary skill in the art. For example, when two different excipients are combined, they are preferably combined in 50:50, 60:40, 70:30, 80:20, or 90:10 ratios. When three different excipients are combined, they are preferably combined in ratios of 80:10:10, 70:20:10, 60:30:10, 60:20:20, 50:40:10, 50:30:20, or 40:40:20. It should be understood, however, that any ratio of two or more different excipients may be suitably combined for use in the instant formulations.

In specific formulation embodiments, the therapeutic agent is dissolved in the excipient at a concentration sufficient to provide a therapeutically effective dose of the agent to a subject being treated with the formulation. As will be described in more detail below, the formulations are preferably delivered by an in-dwelling catheter in the subject using a highly reliable delivery device, such as a precision pumping device. In preferred embodiments, the therapeutic agent is therefore dissolved in the excipient at a concentration of at least 0.1 mg/mL, at least 0.3 mg/mL, at least 0.5 mg/mL, at least 1 mg/mL, at least 3 mg/mL, at least 5 mg/mL, at least 10 mg/mL, at least 30 mg/mL, at least 50 mg/mL, or at even higher concentrations.

According to some preferred formulation embodiments,
the therapeutic agent is docetaxel, the excipient comprises a polyethylene glycol and/or a polysorbate nonionic surfactant, and the therapeutic agent is dissolved in the excipient at a concentration of at least 10 mg/mL;
the therapeutic agent is docetaxel, the excipient comprises a polyethoxylated castor oil, a polyethylene glycol, and/or dimethylacetamide, and the therapeutic agent is dissolved in the excipient at a concentration of at least 10 mg/mL;
the therapeutic agent is docetaxel, the excipient comprises a polyethoxylated stearic acid, a polyethylene glycol, and/or dimethylacetamide, and the therapeutic agent is dissolved in the excipient at a concentration of at least 10 mg/mL;
the therapeutic agent is etoposide, the excipient comprises a polyethoxylated castor oil and a polyethylene glycol, and the therapeutic agent is dissolved in the excipient at a concentration of at least 30 mg/mL;
the therapeutic agent is etoposide, the excipient comprises a polyethylene glycol and/or a polysorbate nonionic surfactant, and the therapeutic agent dissolved in the excipient at a concentration of at least 30 mg/mL;
the therapeutic agent is etoposide, the excipient comprises a polyethoxylated castor oil, a polyethylene glycol, and/or dimethylacetamide, and the therapeutic agent is dissolved in the excipient at a concentration of at least 30 mg/mL;
the therapeutic agent is etoposide, the excipient comprises a polyethoxylated stearic acid, a polyethylene glycol, and/or dimethylacetamide, and the therapeutic agent is dissolved in the excipient at a concentration of at least 30 mg/mL;
the therapeutic agent is methotrexate, the excipient comprises a polyethoxylated castor oil, a polyethylene glycol, and/or dimethylacetamide, and the therapeutic agent is dissolved in the excipient at a concentration of at least 10 mg/mL;
the therapeutic agent is methotrexate, the excipient comprises a polyethoxylated stearic acid, a polyethylene glycol, and/or dimethylacetamide, and the therapeutic agent is dissolved in the excipient at a concentration of at least 10 mg/mL;
the therapeutic agent is paclitaxel, the excipient comprises a polyethylene glycol and/or a polysorbate nonionic surfactant, and the therapeutic agent is dissolved in the excipient at a concentration of at least 5 mg/mL;
the therapeutic agent is paclitaxel, the excipient comprises a polyethoxylated castor oil, a polyethylene glycol, and/or dimethylacetamide, and the therapeutic agent is dissolved in the excipient at a concentration of at least 50 mg/mL;
the therapeutic agent is paclitaxel, the excipient comprises a polyethoxylated stearic acid, a polyethylene glycol, and/or dimethylacetamide, and the therapeutic agent is dissolved in the excipient at a concentration of at least 50 mg/mL;
the therapeutic agent is vinblastine, the excipient comprises a polyethoxylated castor oil and a polyethylene glycol, and the therapeutic agent is dissolved in the excipient at a concentration of at least 3 mg/mL;
the therapeutic agent is vinblastine, the excipient comprises a polyethylene glycol and/or a polysorbate nonionic surfactant, and the therapeutic agent is dissolved in the excipient at a concentration of at least 3 mg/mL;
the therapeutic agent is vinblastine, the excipient comprises a polyethoxylated castor oil, a polyethylene glycol, and/or dimethylacetamide, and the therapeutic agent is dissolved in the excipient at a concentration of at least 5 mg/mL; or
the therapeutic agent is vinblastine, the excipient comprises a polyethoxylated stearic acid, a polyethylene glycol, and/or dimethylacetamide, and the therapeutic agent is dissolved in the excipient at a concentration of at least 5 mg/mL.

In highly preferred formulation embodiments, the formulation is suitable for direct delivery to the subject without dilution of the formulation into an aqueous delivery solution prior to administration of the formulation to the subject. As will be described in more detail below, such formulations are particularly suitable for direct delivery to the subject by in-dwelling catheter using a precision delivery device, such as a precision pumping device.

The formulations of the instant disclosure may still further comprise one or more pharmaceutically acceptable agents in addition to the therapeutic agent and the excipient. Such agents may act, for example, to stabilize or increase the bioavailability of the active therapeutic agent. Such physiologically acceptable agents include, for example, carbohydrates, such as glucose, sucrose, or dextrans, antioxidants, such as ascorbic acid or glutathione, chelating agents, low molecular weight proteins, buffering agents, anti-microbial agents, or the like, as are typically employed in pharmaceutical formulations. See Remington: The Science and Practice of Pharmacy, 20th ed. (Alfonso R. Gennaro ed.), 2000.

The phrase “pharmaceutically acceptable” is employed herein to refer to those compounds, materials, compositions, and/or dosage forms that are, within the scope of sound medical judgment, suitable for use in contact with the tissues of human beings and animals without excessive toxicity, irritation, allergic response, or other problem or complication, commensurate with a reasonable benefit/risk ratio.

### Systems for Delivery of Therapeutic Agents

The formulations of the instant disclosure are particularly suitable for use in delivering a therapeutic agent to a subject in need of treatment without prior dilution of the agent in aqueous or other solution. In order for the therapeutic agent to be delivered in this manner, the formulation should be delivered to a location in the subject's body where the formulation is rapidly diluted and efficiently mixed with a large volume of the subject's blood. The therapeutic agent is thereby quickly dissolved in the bloodstream and distributed throughout the subject's body by the delivery system.

In order to achieve proper dilution and mixing in the subject's body, the formulation is advantageously delivered by an in-dwelling catheter using a delivery device such as a precision pumping device. In this aspect of the invention, the disclosure therefore provides systems for the delivery of a therapeutic agent comprising a therapeutic formulation, for example one of the above-described formulations comprising a therapeutic agent and an excipient, and a delivery device, wherein the system is configured for direct delivery of the formulation by a catheter to a subject in need of therapeutic treatment.

Delivery devices suitable for use in the instant systems include any device capable of delivering the instant formulations at reliable and steady rates over sufficiently long periods of time, so as to provide suitable therapeutic levels of the therapeutic agent in the subject's body during those periods. Pumps include forced extrusion pumps, such as those typically used for the delivery of insulin in diabetic patients. Extrusion pumps are available commercially from a wide variety of sources, including Animas, Medtronic Omnipod, Roche, Tandem, and others. Ideally, the pumps are capable of delivering volumes in the range of 1 μL to 1000 μL per hour in a continuous manner over the full period of treatment.

In some embodiments, the systems are designed to use exchangeable reservoirs for delivery of the therapeutic agent. Such exchangeable reservoirs, for example exchangeable cartridges, enable the delivery of larger volumes of the therapeutic agent during a treatment regimen than would otherwise be possible using a non-exchangeable reservoir.

In some embodiments, the system is configured to deliver the formulation to the subject at no more than 1 mL per hour, no more than 0.5 mL per hour, no more than 0.2 mL per hour, no more than 0.1 mL per hour, or at even lower rates.

In some embodiments, the system is configured to deliver at least a therapeutic dose of the therapeutic agent to the subject for at least 12 hours, for at least 18 hours, for at least 24 hours, for at least 36 hours, for at least 72 hours, or for even longer periods. Ideally, the dosing obtained using the disclosed systems is similar to the delivery levels achieved with standard infusion methodologies for a given therapeutic agent using traditional delivery systems.

In some embodiments, the system is configured to deliver less than a toxic dose of the therapeutic agent to the subject for at least 12 hours, for at least 18 hours, for at least 24 hours, for at least 36 hours, for at least 72 hours, or for even longer periods. Ideally, the dosing obtained using the disclosed systems provides less toxic levels of delivery than observed with standard infusion methodologies for a given therapeutic agent using traditional delivery systems.

In some embodiments, the system is configured to deliver at least a therapeutic dose and less than a toxic dose of the therapeutic agent to the subject for at least 12 hours, for at least 24 hours, for at least 36 hours, for at least 72 hours, or for even longer periods.

The catheter used to deliver the therapeutic agents in the instant systems is advantageously an in-dwelling catheter positioned so that the formulation being delivered is rapidly diluted and mixed with the blood of the subject being treated using the system. In some embodiments, the catheter is a peripherally inserted central catheter, a Hickman line, a Broviac catheter, a Groshong catheter, or a central venous catheter. In preferred embodiments, the catheter is a peripherally inserted central catheter. The proper placement of such catheters in the blood vessels of a subject in need of treatment is well understood by those of ordinary skill in the art.

The instant systems provide advantages over the current systems for the delivery of therapeutic agents in patients, for example for the delivery of chemotherapeutic agents in cancer patients. Current systems typically involve the use of “infusions” or “infusion therapy”. These terms refer to the delivery of medications directly into the veins of a patient, for example by intravenous administration. Such delivery cannot typically be performed by the patient herself without the presence of a medical provider. Accordingly, the administration is typically performed in an office-based setting, for example at an “infusion center”.

Many infusion medications are prepared at the time of treatment by a medical professional, commonly a registered nurse. If the medication is delivered intravenously, it will likely be prepared and added to an appropriately sized bag of sterile aqueous solution, which is then administered through an intravenous catheter placed by the registered nurse. Some intravenous and injectable medications come in pre-prepared forms that may not require as much advanced preparation, although the low solubility of most chemotherapeutic agents in traditional aqueous infusion vehicles requires large dilutions of the agents into large volumes of intravenous fluids, and the slow infusion of those large volumes into the subject during the course of treatment. Such administration techniques are unnecessary using the therapeutic delivery systems of the instant disclosure.

### Methods for Delivery of Therapeutic Formulations

In another aspect, the instant disclosure provides methods of therapy comprising the steps of delivering a therapeutic formulation to a subject in need thereof, wherein the therapeutic formulation is any of the above-described formulations, and the therapeutic agent is delivered to the subject by a catheter using a delivery device.

In preferred embodiments, the agent is administered continuously. The duration of administration of the therapeutic agent, e.g., the period of time over which the agent is administered, may vary, depending on any of a variety of factors, e.g., the formulation of the agent, the patient response, and so forth. For example, the agent may be administered over a period of time of at least 5 minutes, at least 30 minutes, at least one hour, at least 2 hours, at least 4 hours, at least 8 hours, at least one day, at least one week, or even longer. In other embodiments, the agent may be administered over a period of time of no more than one week, no more than one day, no more than 8 hours, no more than 4 hours, no more than 2 hours, no more than one hour, no more than 30 minutes, no more than 5 minutes, or even shorter. In some embodiments, the agent may be administered for a time period of about 5 minutes to 30 minutes, of about 30 minutes to one hour, of about one hour to 2 hours, of about 2 hours to 4 hours, of about 4 hours to 8 hours, of about 8 hours to one day, or of about one day to one week.

In some embodiments, the delivery device is a precision pumping device, such as a forced extrusion pump. As described above, such pumps are commonly used in the delivery of insulin to diabetic patients.

In some embodiments, the formulation is delivered to the subject at no more than 1 mL per hour.

In some embodiments, at least a therapeutic dose of the therapeutic agent is delivered to the subject for at least 12 hours. In other embodiments, less than a toxic dose of the therapeutic agent is delivered to the subject for at least 12 hours. In still other embodiments, at least a therapeutic dose and less than a toxic dose of the therapeutic agent is delivered to the subject for at least 12 hours.

In specific embodiments, the catheter is a peripherally inserted central catheter, a Hickman line, a Broviac catheter, a Groshong catheter, or a central venous catheter. Most preferably, the catheter is a peripherally inserted central catheter.

In some embodiments, the delivery is by steady-state delivery.

In some embodiments, the delivery reduces the incidence of neutropenia in the subject.

In some embodiments, the subject is a mammalian subject. More specifically, the subject is a human subject.

The therapeutic agents described herein can be administered alone or can be coadministered to the individual. Coadministration is meant to include simultaneous or sequential administration of the compounds individually or in combination (more than one compound). Thus, the preparations can also be combined, when desired, with other active substances related to the treatment of a specified condition (e.g., to reduce metabolic degradation).

It will be readily apparent to one of ordinary skill in the relevant arts that other suitable modifications and adaptations to the formulations, systems, and methods described herein may be made without departing from the scope of the invention or any embodiment thereof. Having now described the present invention in detail, the same will be more clearly understood by reference to the following Example, which is included herewith for purposes of illustration only and is not intended to be limiting of the invention.

### EXAMPLE

In order to deliver a cytolytic or other therapeutic agent in a continuous dosing strategy using a forced extrusion pump, the agent must be solubilized at a high concentration to achieve similar dosing to current approved forms over long periods of time. This example is designed to determine formulations comprising a therapeutic agent and an excipient that are suitable for this purpose for each agent.

Eleven compounds and seven solubilizing excipients were obtained from commercial sources (see Table 1). The compounds represent the most commonly prescribed cytolytic medications on the market, excluding compounds such as anthracyclines (which have been shown to be no less toxic given continuously) and cyclophosphamide (which requires too high a dose to be feasibly delivered in this manner). The excipients listed comprise all of the organic solubilizers currently approved by the FDA for intravenous use in human patients.

TABLE 1
List of compounds and Excipients tested in this example

| Compounds | Excipients |
|---|---|
| Cisplatin | Cremophor EL |
| Carboplatin | Cremophor ELP |
| Oxaliplatin | Solutol HS-15 |
| Paclitaxel | PEG400 |
| Docetaxel | PEG300 |
| Vinblastine | Ethanol |
| gemcitabine hydrochloride | dimethyl acetamide |
| fluorouracil (5-FU) | |
| Irinotecan | |
| Methotrexate | |
| Etoposide | |

Concentration targets were calculated based on providing the same amount of drug used clinically over the time period typically allotted for each compound (see Table 2 for target concentrations). Each solubilizing mixture was started at the target concentration, and if ineffective, diluted 2-fold successively until solubilization was achieved (sometimes with the help of gentle heating, mixing, or sonication), or until the solution was tested at 1:16 the target concentration, in which case further testing was halted (this concentration is approximately what would be required to hit a target dose with a daily cartridge exchange in a delivery device).

TABLE 2
Target concentrations for each agent

| | | |
|---|---|---|
| carboplatin | 32.1 | mg/mL |
| oxaliplatin | 15.2 | mg/mL |
| Paclitaxel | 62.5 | mg/mL |
| Docetaxel | 11.9 | mg/mL |
| vinblastine | 6.67 | mg/mL |
| gemcitabine hydrochloride | 352 | mg/mL |
| fluorouracil (5-FU) | 90 | mg/mL |
| irinotecan | 64.3 | mg/mL |
| methotrexate | 10.7 | mg/mL |
| Etoposide | 125 | mg/mL |

Three primary formulation combinations were tested in this example:
Solvent and co-solvent systems (PEG400 and Tween® 20 or Tween® 80)
Surfactant and co-solvent systems (Cremophor® or Solutol® and PEG400)
Surfactant and solvent/co-solvent systems (Cremophor® or Solutol® and DMA or ethanol and PEG400)
   For the first two combinations, mixtures were made upfront and solubility of each compound tested within. For the third, the compound was pre-dissolved in either DMA or ethanol and the remaining components then added as a mixture.

### Results

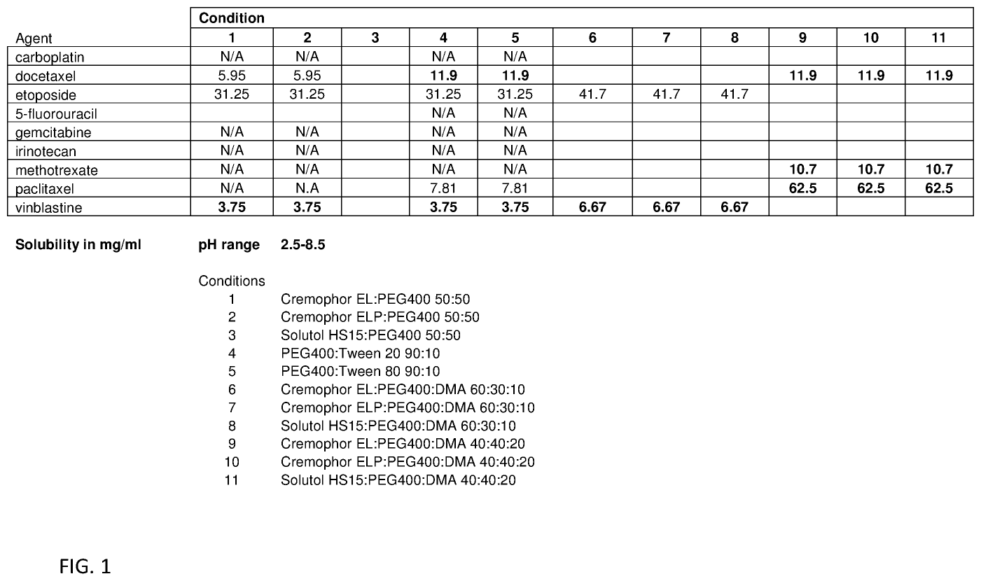

FIG. 1 shows the systems tested, and concentrations achieved, for each combination. None of the compounds were sufficiently soluble in ethanol for these experiments. DMA was sufficient to solubilize docetaxel, paclitaxel, methotrexate, vinblastine, and etoposide. Multiple examples were established for each organic blend tested, with surfactants and solvent/cosolvent systems being the most effective. Several of the compounds were more polar than the others, and therefore insoluble in all of the organic blends tested (these included carboplatin, irinotecan, 5-FU, and gemcitabine hydrochloride). Of these, increased solubility of gemcitabine can be achieved with the free base instead of the salt.

All patents, patent publications, and other published references mentioned herein are hereby incorporated by reference in their entireties as if each had been individually and specifically incorporated by reference herein.

While specific examples have been provided, the above description is illustrative and not restrictive. Any one or more of the features of the previously described embodiments can be combined in any manner with one or more features of any other embodiments in the present invention. Furthermore, many variations of the invention will become apparent to those skilled in the art upon review of the specification. The scope of the invention should, therefore, be determined by reference to the appended claims, along with their full scope of equivalents.

What is claimed is:

1. A therapeutic formulation comprising:
a therapeutic agent and an excipient,
wherein the formulation is suitable for direct delivery at high concentration by a catheter to a subject in need of therapeutic treatment.

2. The formulation of claim 1, wherein the therapeutic agent is a chemotherapeutic agent.

3. The formulation of claim 2, wherein the chemotherapeutic agent is an alkylating agent, an anthracycline, a cytoskeletal disruptor, an epothilone, a histone deacetylase inhibitor, a kinase inhibitor, a nucleotide analogue or precursor, a peptide, a platinum-based agent, a retinoid, a topoisomerase inhibitor, or a vinca alkaloid.

4. The formulation of claim 3, wherein the chemotherapeutic agent is paclitaxel, taxol, docetaxel, taxotere, cisplatin, carboplatin, oxaliplatin, etoposide, vincristine, cyclophosphamide, methotrexate, fluorouracil, gemcitabine, topotecan, irinotecan, melphalan, or doxorubicin.

5. The formulation of claim 1, wherein the excipient increases the solubility of a hydrophobic therapeutic agent in the formulation.

6. The formulation of claim 5, wherein the excipient comprises dimethylacetamide, ethanol, a polyethylene glycol, propylene glycol, a polyethoxylated nonionic surfactant, a polysorbate nonionic surfactant, or a cyclodextrin.

7. The formulation of claim 1, wherein the excipient increases the solubility of a hydrophilic therapeutic agent in the formulation.

8. The formulation of claim 7, wherein the excipient comprises an acid, a base, or a salt.

9. The formulation of claim 1, wherein the therapeutic agent is dissolved in the excipient at a concentration of at least 1 mg/mL, at least 3 mg/mL, at least 5 mg/mL, at least 10 mg/mL, at least 30 mg/mL, or least 50 mg/mL.

10. The formulation of claim 1, wherein the therapeutic agent is docetaxel, the excipient comprises a polyethoxylated castor oil and/or a polyethylene glycol, and the therapeutic agent is dissolved in the excipient at a concentration of at least 5 mg/mL.

11. The formulation of claim 1, wherein the therapeutic agent is docetaxel, the excipient comprises a polyethylene glycol and/or a polysorbate nonionic surfactant, and the therapeutic agent is dissolved in the excipient at a concentration of at least 10 mg/mL.

12. The formulation of claim 1, wherein the therapeutic agent is docetaxel, the excipient comprises a polyethoxylated castor oil, a polyethylene glycol, and/or dimethylacetamide, and the therapeutic agent is dissolved in the excipient at a concentration of at least 10 mg/mL.

13. The formulation of claim 1, wherein the therapeutic agent is docetaxel, the excipient comprises a polyethoxylated stearic acid, a polyethylene glycol, and/or dimethylacetamide, and the therapeutic agent is dissolved in the excipient at a concentration of at least 10 mg/mL.

14. The formulation of claim 1, wherein the therapeutic agent is etoposide, the excipient comprises a polyethoxylated castor oil and a polyethylene glycol, and the therapeutic agent is dissolved in the excipient at a concentration of at least 30 mg/mL.

15. The formulation of claim 1, wherein the therapeutic agent is etoposide, the excipient comprises a polyethylene glycol and/or a polysorbate nonionic surfactant, and the therapeutic agent is dissolved in the excipient at a concentration of at least 30 mg/mL.

16. The formulation of claim 1, wherein the therapeutic agent is etoposide, the excipient comprises a polyethoxylated castor oil, a polyethylene glycol, and/or dimethylacetamide, and the therapeutic agent is dissolved in the excipient at a concentration of at least 30 mg/mL.

17. The formulation of claim 1, wherein the therapeutic agent is etoposide, the excipient comprises a polyethoxylated stearic acid, a polyethylene glycol, and/or dimethylacetamide, and the therapeutic agent is dissolved in the excipient at a concentration of at least 30 mg/mL.

18. The formulation of claim 1, wherein the therapeutic agent is methotrexate, the excipient comprises a polyethoxylated castor oil, a polyethylene glycol, and/or dimethylacetamide, and the therapeutic agent is dissolved in the excipient at a concentration of at least 10 mg/mL.

19. The formulation of claim 1, wherein the therapeutic agent is methotrexate, the excipient comprises a polyethoxylated stearic acid, a polyethylene glycol, and/or dimethylacetamide, and the therapeutic agent is dissolved in the excipient at a concentration of at least 10 mg/mL.

20. The formulation of claim 1, wherein the therapeutic agent is paclitaxel, the excipient comprises a polyethylene glycol and/or a polysorbate nonionic surfactant, and the therapeutic agent is dissolved in the excipient at a concentration of at least 5 mg/mL.

21. The formulation of claim 1, wherein the therapeutic agent is paclitaxel, the excipient comprises a polyethoxylated castor oil, a polyethylene glycol, and/or dimethylacetamide, and the therapeutic agent is dissolved in the excipient at a concentration of at least 50 mg/mL.

22. The formulation of claim 1, wherein the therapeutic agent is paclitaxel, the excipient comprises a polyethoxylated stearic acid, a polyethylene glycol, and/or dimethylacetamide, and the therapeutic agent is dissolved in the excipient at a concentration of at least 50 mg/mL.

23. The formulation of claim 1, wherein the therapeutic agent is vinblastine, the excipient comprises a polyethoxylated castor oil and a polyethylene glycol, and the therapeutic agent is dissolved in the excipient at a concentration of at least 3 mg/mL.

24. The formulation of claim 1, wherein the therapeutic agent is vinblastine, the excipient comprises a polyethylene glycol and/or a polysorbate nonionic surfactant, and the therapeutic agent is dissolved in the excipient at a concentration of at least 3 mg/mL.

25. The formulation of claim 1, wherein the therapeutic agent is vinblastine, the excipient comprises a polyethoxylated castor oil, a polyethylene glycol, and/or dimethylacetamide, and the therapeutic agent is dissolved in the excipient at a concentration of at least 5 mg/mL.

26. The formulation of claim 1, wherein the therapeutic agent is vinblastine, the excipient comprises a polyethoxylated stearic acid, a polyethylene glycol, and/or dimethylacetamide, and the therapeutic agent is dissolved in the excipient at a concentration of at least 5 mg/mL.

27. The formulation of claim 1, wherein the formulation is suitable for direct delivery to the subject without dilution.

28. The formulation of claim 27, wherein the formulation is suitable for direct delivery to the subject without dilution into an aqueous solution.

29. A system comprising:
the formulation of any one of claims 1 to 28; and
a delivery device,
wherein the system is configured for direct delivery of the formulation by a catheter to a subject in need of therapeutic treatment.

30. The system of claim 29, wherein the delivery device is a precision pumping device.

31. The system of claim 29, wherein the formulation is delivered from an exchangeable reservoir.

32. The system of claim 29, wherein the system is configured to deliver the formulation to the subject at no more than 1 mL per hour.

33. The system of claim 29, wherein the system is configured to deliver at least a therapeutic dose of the therapeutic agent to the subject for at least 12 hours.

34. The system of claim 29, wherein the system is configured to deliver less than a toxic dose of the therapeutic agent to the subject for at least 12 hours.

35. The system of claim 29, wherein the system is configured to deliver at least a therapeutic dose and less than a toxic dose of the therapeutic agent to the subject for at least 12 hours.

36. The system of claim 29, wherein the catheter is a peripherally inserted central catheter, a Hickman line, a Broviac catheter, a Groshong catheter, or a central venous catheter.

37. The system of claim 29, wherein the delivery is by steady-state delivery.

38. The system of claim 29, wherein the delivery reduces the incidence of neutropenia in the subject.

39. The system of claim 29, wherein the subject is a human subject.

40. A method of treatment comprising the steps of:
delivering a therapeutic formulation to a subject in need thereof, wherein
the therapeutic formulation is the formulation of any one of claims 1 to 28, and the formulation is delivered directly to the subject by a catheter using a delivery device.

41. The method of claim 40, wherein the delivery device is a precision pumping device.

42. The method of claim 40, wherein the formulation is delivered to the subject at no more than 1 mL per hour.

43. The method of claim 40, wherein at least a therapeutic dose of the therapeutic agent is delivered to the subject for at least 12 hours.

44. The method of claim 40, wherein less than a toxic dose of the therapeutic agent is delivered to the subject for at least 12 hours.

45. The method of claim 40, wherein at least a therapeutic dose and less than a toxic dose of the therapeutic agent is delivered to the subject for at least 12 hours.

46. The method of claim 40, wherein the catheter is a peripherally inserted central catheter, a Hickman line, a Broviac catheter, a Groshong catheter, or a central venous catheter.

47. The method of claim 40, wherein the delivery is by steady-state delivery.

48. The method of claim 40, wherein the delivery reduces the incidence of neutropenia in the subject.

49. The method of claim 40, wherein the subject is a human subject.
